(19) United States
(12) Patent Application Publication
(10) Pub. No.: US 2022/0032089 A1
(43) Pub. Date: Feb. 3, 2022

(54) ULTRASOUND VISUALIZATION, AND ASSOCIATED SYSTEMS AND METHODS

(71) Applicant: CEREVAST MEDICAL, INC., Bothell, WA (US)

(72) Inventor: Francesco P. Curra, Brier, WA (US)

(73) Assignee: CEREVAST MEDICAL, INC., Bothell, WA (US)

(21) Appl. No.: 16/945,314

(22) Filed: Jul. 31, 2020

(51) Int. Cl. A61N 7/00; G01S 15/89; G06N 20/00

(52) U.S. Cl. CPC A61N 7/00; G01S 15/8988; A61N 2007/0052; G06N 20/00; G01S 15/8918

(57) ABSTRACT

Presented herein is ultrasound visualization and associated systems and methods. In one embodiment, a method for an ultrasound imaging, includes: transmitting an imaging ultrasound toward a target tissue; receiving ultrasound echoes of the imaging ultrasound using a first set of receiving beamformer parameters; composing a first two-dimensional (2D) B-mode image of the target tissue based on received ultrasound echoes of the imaging ultrasound; transmitting a therapy ultrasound toward a target tissue by a therapy transducer using a second set of transmitting beamformer parameters; receiving ultrasound echoes of the therapy ultrasound using the first set of receiving beamformer parameters; composing a second 2D B-mode image of the target tissue based on received ultrasound echoes of the therapy ultrasound; and comparing the first 2D B-mode image of the target tissue with the second 2D B-mode image of the target tissue.

### BACKGROUND

Image-guided ultrasound therapy systems typically include a processing unit coupled with one or more ultrasound probes. Dedicated therapy and imaging probes of different characteristics can be used for therapy delivery and imaging feedback in the case of high-intensity focused ultrasound (HIFU) applications. Furthermore, a single ultrasound probe can alternately function for ultrasound therapy and imaging in the case of low-intensity focused (LIFU) applications.

In operation, the imaging portion of the system is intended to generate a traditional ultrasound image of the internal structures of the body and thus provide a frame of reference to “guide” the ultrasound therapy portion. The therapy targets are either pre-programmed or directly selected from two-dimensional (2D) B-mode images and the therapy ultrasound pulses are directed and focused at the selected locations. As a result, the focused ultrasound therapy is fundamentally applied blindly, in a sense that the necessary time delays for focusing the beams are computed purely on a geometrical basis with an assumed constant speed of sound of 1540 m/s through the tissue. This process does not take in consideration possible beam diffraction and refraction effects due to changes in acoustic impedances between different tissues, which may result in the beam deviating from the intended target. This is of particular relevance in presence or proximity of hard structures, such as bones, or in the areas where safeguarding sensitive nerve clusters is important.

To overcome some of these shortfalls, several methods have been developed for guidance and monitoring of ultrasound therapy. Some examples of these approaches are magnetic resonance imaging (MRI) and acoustic simulation-based pre-treatment planning. Both methods are applied in HIFU ablative protocols, where simulation pre-treatment planning is intended to optimize the ultrasound targeting and MRI thermometry is used to monitor and provide feedback during the treatment. In the treatment planning phase, computed tomography (CT) images of the region of interest are acquired, segmented, and mapped by their acoustic properties. Next, ultrasound simulation software is used to model the acoustic wave propagation from virtual point sources located at the desired target points back to the transducer. The received simulated signals are time-reversed and the time delays of each individual element in the probe are computed. The computed time delays for each target are then programmed into the therapy system for treatment. This time reversal approach guaranties proper phase aberration correction and good focusing at the target. MRI thermometry is used to map the temperature profile with high spatial resolution during the treatment and allows monitoring for over/under treatment.

While these clinically employed methods are quite effective in ultrasound therapy systems, they are not real-time and they require additional resources such as modeling software, access to CT Imaging, MRI compatible ultrasound probes, and even a dedicated MRI system. Therefore, such methods are presently very costly. Furthermore, MRI thermometry becomes ineffective for LIFU treatments where the in-situ temperature changes are relatively small. Accordingly, systems and methods are needed for improved accuracy of targeting of the ultrasound therapy beams.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Briefly, the inventive technology uses a standard ultrasound 2D B-Mode image (also referred to as a 2D or a B-Mode image) that is composed by successive acquisition, processing, and display of multiple adjacent one-dimensional (1D) A-Mode line images (also referred to as 1D or A-Mode images) in the region of interest. For each 1D A-Mode line image, either all the imaging transducer elements, or a subset thereof, transmit an interrogating imaging pulse that is specifically time-delayed and focused along that 1D line. Such time-delaying and focusing of the individual transmit elements is generally referred to as the transmit beamformer. The same or different array elements receive the scattered echo from the target. The received echo signals are then processed, whereby all the individual signals are time delayed and aligned for specific detection over that line and summed up to represent the targeted 1D line. Such time-delaying and focusing of the individual receive elements is generally referred to as the receive beamformer.

The summed signal is envelope detected, and the 1D line may be displayed with brightness proportional to the signal amplitude over a gray colormap. The process is repeated for each 1D line and a full collection of 1D lines obtained by the receive elements forms the 2D image. In some embodiments, the ultrasound systems can acquire 2D images at a rate of 30 to 50 frames per seconds for a nominal 20 cm target depth, thus being able to reconstruct the 2D image essentially in real-time.

A person of ordinary skill would understand that with the present inventive technology the imaging and therapy transducer may be two different units, however the imaging and therapy transducer may also be the same unit that alternatively executes the two functions (therapy function and imaging function). Furthermore, the imaging and/or therapy transducers may be either phased array units or unitary units.

Co-Registration

Once the imaging ultrasound is emitted and the resulting 2D images are acquired, the transmit beamformer of the imaging transducer may be turned off, and the parameters of the receive beamformer of the imaging transducer (or of the one and same transducer that is used for both the imaging and therapy functions) may remain fixed for a duration of one or more subsequent therapy ultrasound pulses. When the therapy ultrasound beam is transmitted toward a target, the reflected ultrasound echo is detected by the receive beamformer of the imaging transducer. For example, after each transmit pulse of the therapy array to a given target point, the same full 2D line-based receive beamformer is enabled for the imaging transducer. The process resulting in the real time 2D frame reconstruction of the therapy beam. Such process that uses the therapy ultrasound to acquire 2D image frames may be referred to as a 2D Therapy Beam Visualization (TBV) mode.

Since the parameters of the receive beamformer are at this point fixed, the acquired TBV 2D image is co-registered with the earlier-acquired 2D image that is based on the standard imaging ultrasound. Different 2D frames may be rendered with different colormaps and overlaid on each other. For example, the 2D image that was initially acquired using imaging ultrasound may be displayed in one color, and the subsequent TBV 2D images that are acquired from the therapy beam echoes may be overlaid in a different color. Since the acquisition of the subsequent TBV 2D images is co-registered with the 2D image that was initially acquired with the imaging ultrasound, the inventive technology can achieve an effective visualization and tracking of the therapy pulse in real time. Visualization of the therapy beam pattern allows a determination of in-situ spatial features of the therapy ultrasound field, and, consequently, provides an assessment of the targeting accuracy and the distribution of acoustic energy in real-time. As discussed above, due to diffraction and refraction effects, a simple reverse-reconstruction of the ultrasound path from the target area may not provide required accuracy of targeting.

With the inventive technology, both the initial 2D B-mode image and the subsequent TBV 2D mode image are based on the same imaging receive beamformer. Therefore, the two 2D B-mode images are co-registered and the TBV beam corresponds to the relative location of the internal tissue structures. As a result, methods and systems of the inventive technology may be immune to motion artifacts when the image acquisition is gated, for example, to the heartbeat or respiration. Furthermore, a person skilled in the art, would recognize that the proposed inventive technology is independent of the type of beamforming and frame reconstructions methods. For example, even though the inventive technology was described with reference to standard line-based 2D imaging, flash imaging and pixel-based reconstruction and/or variations thereof including, without restrictions, coded-excitation, pulse sequencing and inversion, and harmonic approaches are also applicable in this context.

Artificial intelligence (AI)

In some embodiments, the therapy beam targeting may be optimized based on artificial intelligence (AI). For example, an underlining neural network engine may iteratively minimize differences between the actual therapy beam pattern spatial pattern and the trained refence theoretical or simulated beam patterns for a given transducer. In some embodiments, the TBV 2D frame is fed to the neural network that uses transducer template beam patterns as training sets. For example, for a phased array therapy transducer, the AI engine may produce new element-by-element time delays that minimize differences between the actual beam patter acquired by the TBV 2D frame and a desired beam pattern. The process may repeat with the new TBV 2D frame obtained with new element-by-element time delays until a convergence is achieved to a target tolerance. Considering that a standard ultrasound imaging system can acquire and process on average 30 to 50 frames per second, such convergence may be relatively fast. Additionally, all the updated data (e.g., TBV 2D images, element-by-element time delays, etc., that are fed to the AI engine) continuously enhance the training set and machine learning features of the AI engine. As a result, the described method may perform unbiased, in-situ, and real-time phase aberration corrections.

The inventive technology is generally agnostic to the type of ultrasound probe used. In different embodiments, 1D or 2D linear or phased arrays may be employed. In the case of 2D arrays, the inventive technology may provide the full 3D spatial shape of the therapy beam, which becomes important in the case of possible off-axis side lobes resulting from different designs of the transducers. Additionally, and as explained above, the inventive technology does not require a single ultrasound array for both therapy and imaging, because the inventive technology may also operate with two distinct transducers provided that triggering information between the two probes is shared to perform synchronization.

In one embodiment, a method for 1 an ultrasound imaging, includes: (i) transmitting an imaging ultrasound toward a target tissue; (ii) receiving ultrasound echoes of the imaging ultrasound using a first set of receiving beamformer parameters; (iii) composing a first two-dimensional (2D) B-mode image of the target tissue based on received ultrasound echoes of the imaging ultrasound; (iv) transmitting a therapy ultrasound toward a target tissue using a second set of transmitting beamformer parameters; (v) receiving ultrasound echoes of the therapy ultrasound using the first set of receiving beamformer parameters; (vi) composing a second 2D B-mode image of the target tissue based on received ultrasound echoes of the therapy ultrasound; and (vii) comparing the first 2D B-mode image of the target tissue with the second 2D B-mode image of the target tissue.

In one embodiment, comparing the first 2D B-mode image of the target tissue with the second 2D B-mode image of the target tissue includes: displaying the first 2D B-mode image in a first color; and overlaying the second 2D B-mode image in a second color over the first 2D B-mode image.

In another embodiment, the imaging ultrasound is transmitted by an imaging transducer. Furthermore, the ultrasound echoes of the imaging ultrasound are received by the imaging transducer; the therapy ultrasound is transmitted by a therapy transducer; and the ultrasound echoes of the therapy transducer are received by the imaging transducer.

In one embodiment, the therapy transducer is a phased array therapy transducer comprising a plurality of phased array elements. In another embodiment, the image transducer is a phased array therapy transducer that is different from the therapy transducer.

In one embodiment, the imaging ultrasound and the therapy ultrasound are transmitted and received by a same transducer which alternately executes roles of the imaging transducer and the therapy transducer.

In another embodiment, if a match between the first 2D B-mode image and the second 2D B-mode image is within a predetermined threshold match, the method also includes transmitting additional therapy ultrasound toward the target tissue by a therapy transducer using the second set of transmitting beamformer parameters. The method further includes comparing the first 2D B-mode image of the target tissue with additional second 2D B-mode image of the target tissue. In one embodiment, the method also includes adjusting the second set of transmitting beamformer parameters to target another location of the target tissue.

In one embodiment, if a match between the first 2D B-mode image and the second 2D B-mode image is below a predetermined threshold match, the method also includes adjusting the second set of transmitting beamformer parameters of a therapy transducer; and transmitting the therapy ultrasound toward the target tissue by the therapy transducer using the adjusted second set of transmitting beamformer parameters.

In one embodiment, adjusting the second set of transmitting beamformer parameters is performed by an artificial intelligence (AI) engine. In another embodiment, the AI engine utilizes transducer template beam patterns as training sets to produce new element-by-element time delays that minimize differences between the second 2D B-frame and the first 2D B-frame. In one embodiment, adjusting the second set of the transmitting beamformer parameters includes performing real-time phase aberration corrections of the transmitting beamformer parameters by the AI engine.

In one embodiment, the method also includes acquiring new second 2D B-frame images until a convergence to a target tolerance is achieved.

In one embodiment, a computer-readable storage device stores non-volatile computer-executable instructions, which, when executed, cause an ultrasound system to: (i) transmit an imaging ultrasound toward a target tissue; (ii) receive ultrasound echoes of the imaging ultrasound using a first set of receiving beamformer parameters; (iii) compose a first two-dimensional (2D) B-mode image of the target tissue based on received ultrasound echoes of the imaging ultrasound; (iv) transmit a therapy ultrasound toward a target tissue using a second set of transmitting beamformer parameters; (v) receive ultrasound echoes of the therapy ultrasound using the first set of receiving beamformer parameters; (vi) compose a second 2D B-mode image of the target tissue based on received ultrasound echoes of the therapy ultrasound; and (vii) compare the first 2D B-mode image of the target tissue with the second 2D B-mode image of the target tissue.

In one embodiment, the instructions further cause the ultrasound system to: determine whether a match between the first 2D B-mode image and the second 2D B-mode image is within a predetermined threshold match; if the match is within the predetermined threshold match, transmit additional therapy ultrasound toward the target tissue using the second set of transmitting beamformer parameters; and compare the first 2D B-mode image of the target tissue with additional second 2D B-mode image of the target tissue.

In one embodiment, the instructions further cause the ultrasound system to: determine whether a match between the first 2D B-mode image and the second 2D B-mode image is within a predetermined threshold match; if the match is outside of the predetermined threshold match, adjust the second set of transmitting beamformer parameters; transmit the therapy ultrasound toward the target tissue using the adjusted second set of transmitting beamformer parameters; and compare the first 2D B-mode image of the target tissue with additional second 2D B-mode image of the target tissue.

In another embodiment, adjusting the second set of transmitting beamformer parameters is performed by an artificial intelligence (AI) engine.

In one embodiment, the AI engine utilizes transducer template beam patterns as training sets to produce new element-by-element time delays that minimize differences between the second 2D B-frame and the first 2D B-frame.

In one embodiment, the imaging ultrasound is transmitted by an imaging transducer; the ultrasound echoes of the imaging ultrasound are received by the imaging transducer; the therapy ultrasound is transmitted by a therapy transducer; and the ultrasound echoes of the therapy transducer are received by the imaging transducer.

In one embodiment, the imaging ultrasound and the therapy ultrasound are transmitted and received by a same transducer which alternately executes roles of the imaging transducer and the therapy transducer.

### DESCRIPTION OF THE DRAWINGS

The foregoing aspects and many of the attendant advantages of this inventive technology will become more readily appreciated as the same become better understood by reference to the following detailed description, when taken in conjunction with the accompanying drawings, wherein:

FIG. 1 is an isometric diagram of an ultrasound system in accordance with an embodiment of the present technology;

FIG. 2 is an isometric diagram of ultrasound engine and cable in accordance with an embodiment of the present technology;

FIGS. 3A-3C are schematic diagrams of ultrasound phased arrays in accordance with embodiments of the present technology;

FIGS. 4-6 are schematic diagrams of 2D image acquisition in accordance with embodiments of the present technology;

FIG. 7 is a flow chart illustrating an ultrasound method in accordance with embodiments of the present technology;

FIG. 8 is a flow chart illustrating another ultrasound method in accordance with embodiments of the present technology; and

FIGS. 9A-9C are graphs illustrating overlays of the 2D ultrasound images in accordance with an embodiment of the present technology.

### DETAILED DESCRIPTION

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the figures, can be arranged, substituted, combined, separated, and designed in a wide variety of different configurations, all of which are explicitly contemplated herein.

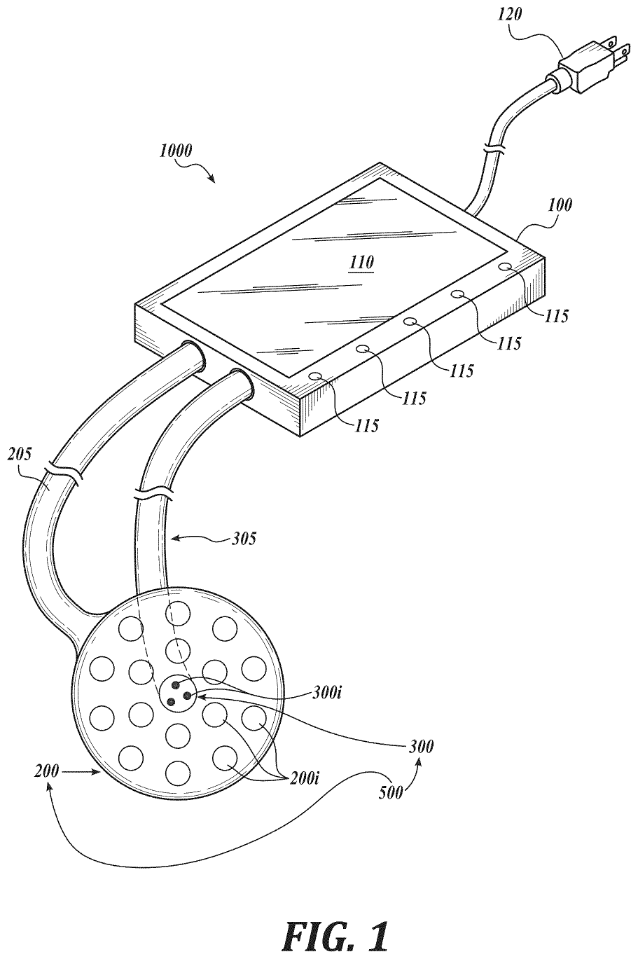

FIG. 1 is an isometric diagram of an ultrasound system 1000 in accordance with an embodiment of the present technology. The ultrasound system 1000 includes an ultrasound probe 500 having a therapy transducer 200 and optionally an imaging transducer 300. In other embodiments, the therapy transducer and the imaging transducer may be the same unit. In different embodiments, the therapy transducer 200 may be a phased array transducer that includes a plurality of phased array elements 200i, and the imaging transducer 300 may be a phased array transducer that includes a plurality of phased array elements 300i.

In operation, the ultrasound probe 500 may be controlled by an ultrasound engine 100 that includes a controller (e.g., a computer, a smart device, etc.) with suitable software, a display 110 and commands 115 for controlling the ultrasound engine. The monitor 110 can display images of the target tissue that are obtained, for example, by an imaging transducer 300 of the ultrasound probe 100. The ultrasound probe 500 may be powered through a power cable 120.

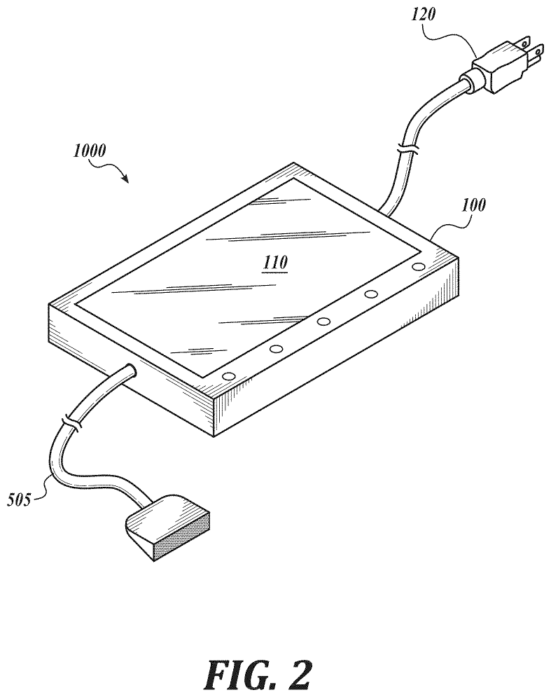

FIG. 2 is an isometric diagram of the ultrasound engine 110 and ultrasound cable 505 in accordance with an embodiment of the present technology. In different embodiments, the ultrasound cable 505 may connect different imaging/therapy transducers with the ultrasound engine 110. Ultrasound beamformers (e.g., transmit and receive beamformers) may reside in the memory of the ultrasound engine or directly on the probe. As explained above, in different embodiments the imaging and therapy transducers may be two different units or one transducer my alternatively execute the therapy function and imaging function. Furthermore, the imaging and/or therapy transducers may be either unitary units or phased array units like those illustrates in FIG. 1.

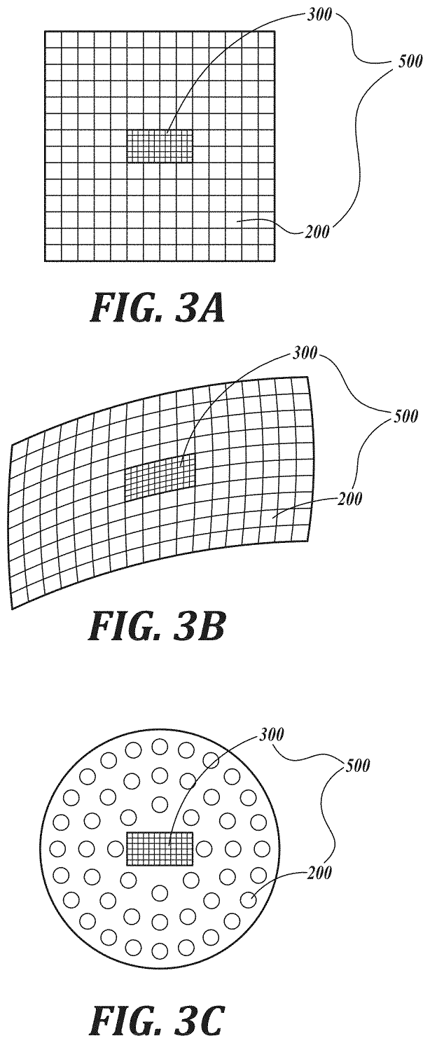

FIGS. 3A-3C are schematic diagrams of ultrasound phased arrays in accordance with embodiments of the present technology. FIG. 3A illustrates a phased ultrasound array 500 having imaging array elements 300 and therapy array elements 200. Illustrated phased ultrasound array 500 has a 2D rectangular shape.

FIG. 3B illustrates a curved phased array 500. FIG. 3C illustrates a circular phased ultrasound array 500 where the therapy array elements 200 are arranged over a circular area and the imaging array elements 300 are arranged over a rectangular area. Other shapes of the ultrasound arrays, both phased arrays and unitary transducers, are also possible in different embodiments.

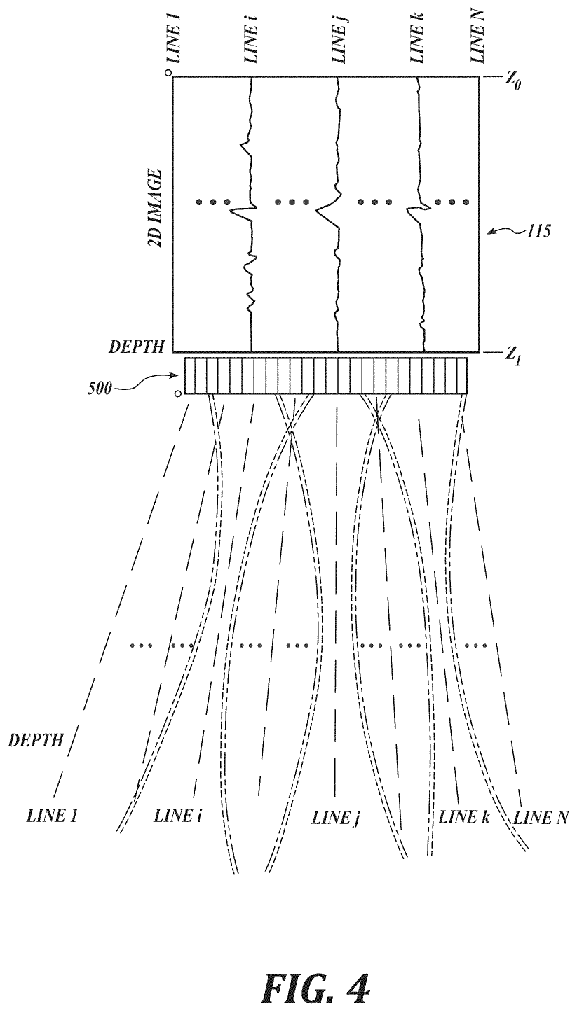

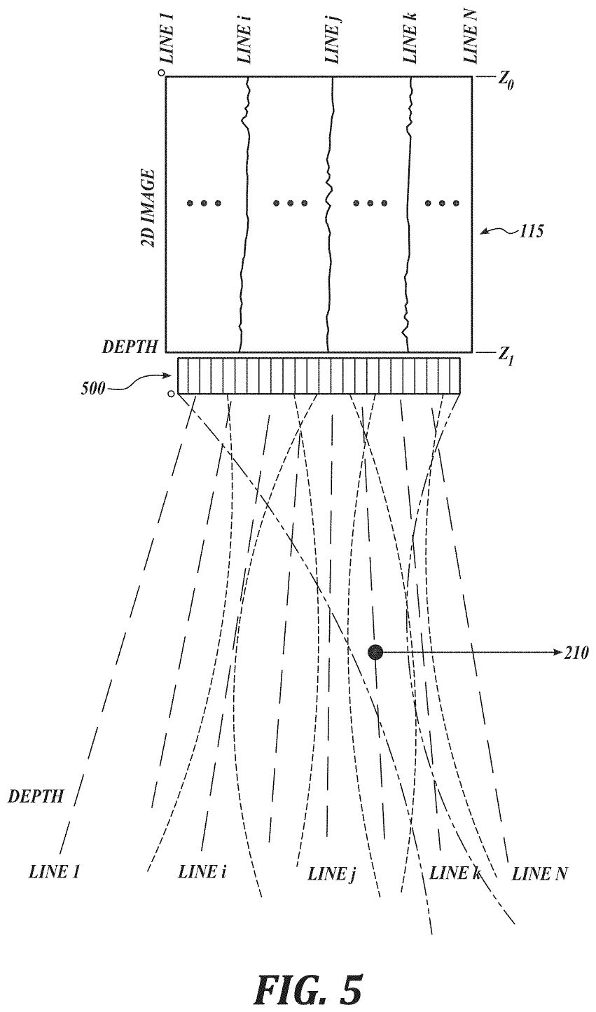

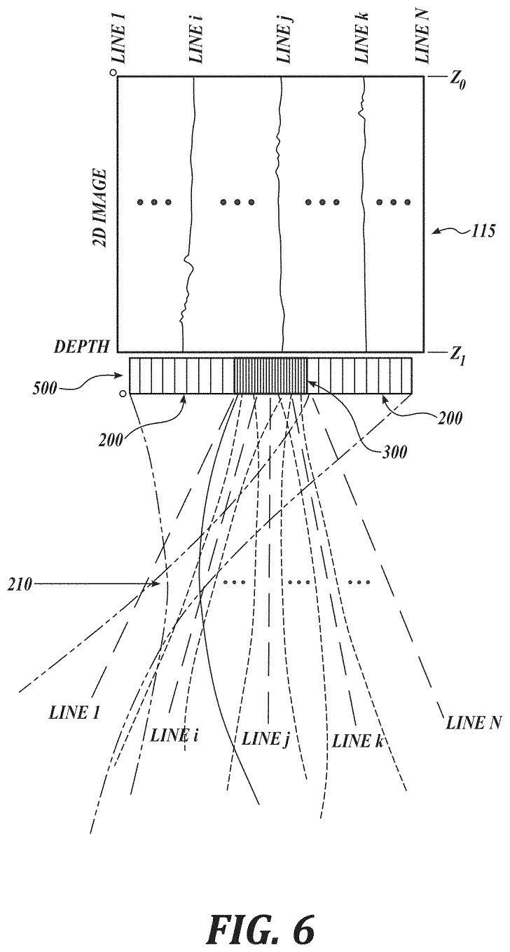

FIGS. 4-6 are schematic diagrams of 2D image acquisition in accordance with embodiments of the present technology. Each schematic diagram of FIGS. 4-6 illustrates an ultrasound phased array 500 that transmits and receives ultrasound signals. FIGS. 4 and 5 illustrate the ultrasound phased array 500 where the elements of the phased array alternately transmit the ultrasound toward a target, and then receive the ultrasound echoes. FIG. 6 illustrates the ultrasound phases array 500 having separate imaging array elements 300 and therapy array elements 200.

In operation, array elements may transmit ultrasound along multiple 1D lines using the transmit beamformer. In the context of this application, the term beamformer encompasses sequential or parallel activation, phase delays, power levels, frequency of oscillation, etc., of the elements of the phased array (or the analogous parameters of a unitary ultrasound transducer). The transmitted ultrasound along 1D lines is represented by the phantom lines in FIGS. 4-6. In operation, the transmitted ultrasound is directed toward a target tissue, in particular toward a focus point or area 210.

The received ultrasound echoes are acquired by a set of same or different elements of the ultrasound phased array 500 by focusing these elements at the target 1D lines using a receive beamformer. The ultrasound echoes are acquired along the dashed lines in FIGS. 4-6. Thus acquired 1D ultrasound echoes are arranged next to each other in a line-by-line fashion to form a 2D displayed image 115. Illustrated displayed image 115 has a depth ranging from Z₀ to Z₁.

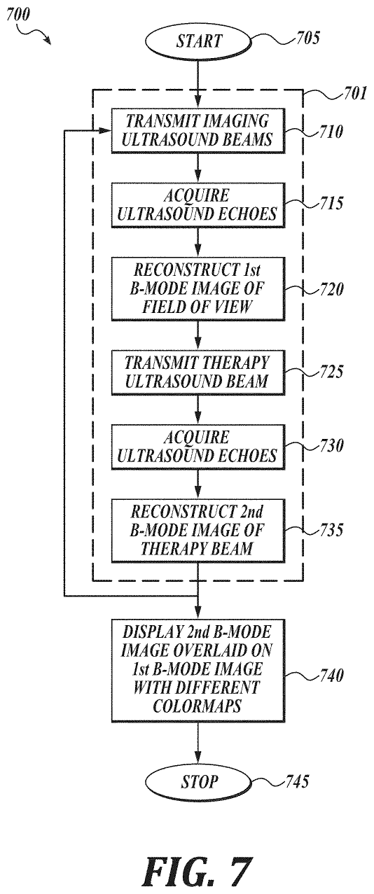

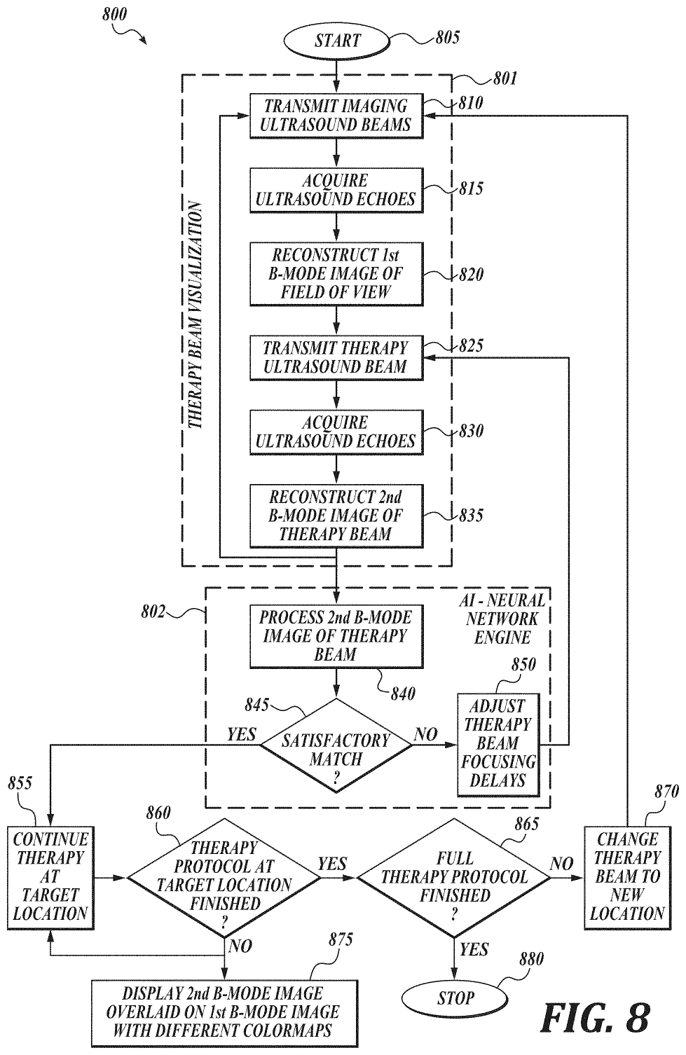

Once the parameters of the receive beamformer are optimized, these parameters may be used for the acquisition of the subsequent 2D ultrasound images as explained in conjunction with FIGS. 7 and 8 below.

FIG. 7 is a flow chart illustrating an ultrasound method in accordance with embodiments of the present technology. In some embodiments, the method may include additional steps or may be practiced without all steps illustrated in the flow chart.

The method starts in block 705. In block 710, imaging ultrasound beams (also referred to as “imaging ultrasound” for brevity and simplicity) is emitted by an ultrasound transmitter, either a dedicated imaging transducer or a therapy transducer that also alternately fulfills the role of the imaging transducer. In block 715, the ultrasound echoes are acquired as a collection of 1D images. The ultrasound echoes are acquired using a first set of receive beamformer parameters.

In block 720, a 2D B-mode image is constructed from the acquired 1D line images. Some embodiments of such B-mode image acquisition are described in conjunction with FIGS. 4-6 above.

In block 725, the therapy transducer transmits therapy ultrasound beam. The therapy ultrasound may be transmitted using a second set of transmit beamformer parameters. In block 730, the ultrasound echoes from the therapy ultrasound are acquired using the set of receive beamformer parameters that is defined in block 715 above.

In block 735, the acquired 2D B-mode image is reconstructed as, for example, an assembly of the 1D A-mode line images. Since the parameters of the receive beamformer are at this point fixed, the reconstructed 2D B-image in block 735, which is based on the therapy ultrasound, is co-registered with the earlier-acquired 2D B-mode in block 720, which is based on the imaging ultrasound. The process may be repeated by starting from block 710 again. Blocks 710-735 may be collectively termed as a therapy beam visualization 701.

In block 740, different 2D B-mode frames may be overlaid. For example, different coloring schemes may be used for the B-modes from blocks 720 and 735. The method ends in block 745.

FIG. 8 is a flow chart illustrating an ultrasound method 800 in accordance with embodiments of the present technology. In some embodiments, the method may include additional steps or may be practiced without all steps illustrated in the flow chart.

Blocks 810-835 of the illustrated therapy beam visualization 801 generally correspond to blocks 710-735 shown in FIG. 7. Next, ultrasound imaging optimization 802 may be performed by the artificial intelligence (AI), also referred to as a neural network engine.

In block 840, the second 2D B-mode image of the therapy beam is processed. For example, this second 2D B-mode image that is based on the ultrasound echoes of the therapy beam may be overlaid over the first 2D B-mode image obtained in blocks 815, 820 that is based on the ultrasound echoes of the imaging beam.

In block 845, a decision is made whether a match is satisfactory between the second 2D B-mode image and the first 2D B-mode image. An unsatisfactory match may indicate, as non-limiting example, an improperly targeted therapy ultrasound away from the target area or a therapy ultrasound that is properly targeted, but lacks desired pressure distribution at the target. Such unsatisfactory match is followed by adjusting therapy beam focusing delays in block 850. Adjusting of the focusing delays may include adjusting the therapy beam beamforming parameters (also referred to as adjusting the second beamforming parameters). These beamforming parameters may rely on AI to iteratively minimize differences between the second 2D B-mode image and the first 2D B-mode image or between the actual therapy beam pattern spatial pattern and the refence theoretical beam pattern for a given transducer.

As explained above, 2D B-mode frames may be fed to the AI that uses transducer template beam patterns as training sets. For a phased array therapy transducer, the AI engine may produce new element-by-element time delays that minimize differences between the actual beam patter acquired in blocks 830, 835 and a desired beam pattern. After the adjustment in block 850, the therapy ultrasound beam is transmitted in block 825, and the process repeats.

If a satisfactory match was achieved in block 845, the therapy continues in block 855 at the target location. In block 860, a decision is made whether a therapy protocol at the target location is finished. For example, the therapy protocol at the target location may be finished after a prescribed number of therapy pulses is reached or after a predetermined therapy time has elapsed. If the therapy protocol at the target location is not finished, the process proceeds to block 875 where the second 2D B-mode image from block 835 is overlaid over the first 2D B-mode image from block 820.

If the therapy protocol at the target location is finished, the process proceeds to block 865 to make a determination whether a full therapy protocol (e.g., including all target location) is finished. If the full therapy protocol is not finished yet, the therapy beam is targeted to a new location, and the process repeats from block 810. If the full therapy protocol is finished, the process stops in block 880.

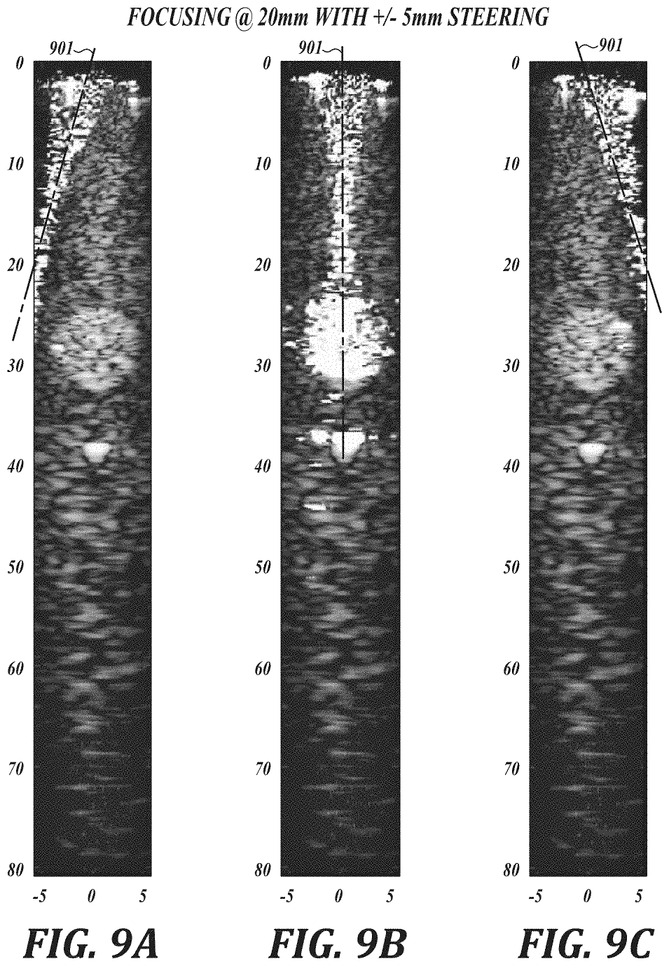

FIGS. 9A-9C are graphs illustrating overlays of the 2D ultrasound images in accordance with an embodiment of the present technology. The horizontal axis represents the width of the 2D ultrasound images in mm. The vertical axis represents the depth of the 2D ultrasound images in mm. For the illustrated graphs, target focusing of the therapy ultrasound is at 20 mm.

Two images are overlaid in each graph. The background image with darker shades was obtained based on the imaging ultrasound echoes (first 2D B-mode ultrasound image). The foreground image with brighter shades was obtained based on the therapy ultrasound echoes (second 2D B-mode ultrasound image). As explained above, the imaging beamformer parameters remain the same for the two images. As a result, a co-registration of the images is achieved.

The three B-mode ultrasound images of FIGS. 9A-9C correspond to different steering 901 of the therapy ultrasound: −5 mm, 0 mm and +5 mm, respectively. For example, the overlay of the first and second 2D B-mode ultrasound images in FIG. 9A indicates that the therapy ultrasound (brighter shades) targeted away and to the left from the round object in the middle of the image at about 30 mm depth. As another example, FIG. 9B indicates that the therapy ultrasound targeted the round object at about 30 mm depth. However, the targeting may still be unsatisfactory if, for example, the pressure distribution in the second 2D B-mode ultrasound image is different from the desired pressure distribution. Under this scenario, the AI engine may adjust the therapy beam beamforming parameters using, for example, the method described in blocks 802 of FIG. 8. The overlay of the first and second 2D B-mode ultrasound images in FIG. 9C indicates that the therapy ultrasound targeted away and to the right from the round object, opposite from the scenario derivable from FIG. 9A.

Many embodiments of the technology described above may take the form of computer- or controller-executable instructions, including routines stored in a non-volatile memory and executed by a programmable computer or controller. Those skilled in the relevant art will appreciate that the technology can be practiced on computer/controller systems other than those shown and described above. The technology can be embodied in a special-purpose computer, controller or data processor that is specifically programmed, configured or constructed to perform one or more of the computer-executable instructions described above. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” etc., mean plus or minus 5% of the stated value.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

What is claimed is:

1. A method for an ultrasound imaging, comprising:
(i) transmitting an imaging ultrasound toward a target tissue;
(ii) receiving ultrasound echoes of the imaging ultrasound using a first set of receiving beamformer parameters;
(iii) composing a first two-dimensional (2D) B-mode image of the target tissue based on received ultrasound echoes of the imaging ultrasound;
(iv) transmitting a therapy ultrasound toward a target tissue using a second set of transmitting beamformer parameters;
(v) receiving ultrasound echoes of the therapy ultrasound using the first set of receiving beamformer parameters;
(vi) composing a second 2D B-mode image of the target tissue based on received ultrasound echoes of the therapy ultrasound; and
(vii) comparing the first 2D B-mode image of the target tissue with the second 2D B-mode image of the target tissue.

2. The method of claim 1, wherein comparing the first 2D B-mode image of the target tissue with the second 2D B-mode image of the target tissue comprises:
displaying the first 2D B-mode image in a first color; and
overlaying the second 2D B-mode image in a second color over the first 2D B-mode image.

3. The method of claim 1, wherein:
the imaging ultrasound is transmitted by an imaging transducer;
the ultrasound echoes of the imaging ultrasound are received by the imaging transducer;
the therapy ultrasound is transmitted by a therapy transducer; and
the ultrasound echoes of the therapy transducer are received by the imaging transducer.

4. The method of claim 1, wherein the therapy transducer is a phased array therapy transducer comprising a plurality of phased array elements.

5. The method of claim 4, wherein the image transducer is a phased array therapy transducer that is different from the therapy transducer.

6. The method of claim 1, wherein:
the imaging ultrasound and the therapy ultrasound are transmitted and received by a same transducer which alternately executes roles of the imaging transducer and the therapy transducer.

7. The method of claim 1, further comprising:
if a match between the first 2D B-mode image and the second 2D B-mode image is within a predetermined threshold match, transmitting additional therapy ultrasound toward the target tissue by a therapy transducer using the second set of transmitting beamformer parameters; and
comparing the first 2D B-mode image of the target tissue with additional second 2D B-mode image of the target tissue.

8. The method of claim 7, further comprising:
adjusting the second set of transmitting beamformer parameters to target another location of the target tissue; and
repeating steps (i)-(vii) of claim 1.

9. The method of claim 1, further comprising:
if a match between the first 2D B-mode image and the second 2D B-mode image is below a predetermined threshold match, adjusting the second set of transmitting beamformer parameters of a therapy transducer; and
transmitting the therapy ultrasound toward the target tissue by the therapy transducer using the adjusted second set of transmitting beamformer parameters.

10. The method of claim 9, wherein adjusting the second set of transmitting beamformer parameters is performed by an artificial intelligence (AI) engine.

11. The method of claim 10, wherein the AI engine utilizes transducer template beam patterns as training sets to produce new element-by-element time delays that minimize differences between the second 2D B-frame and the first 2D B-frame.

12. The method of claim 10, wherein adjusting the second set of the transmitting beamformer parameters comprises performing real-time phase aberration corrections of the transmitting beamformer parameters by the AI engine.

13. The method of claim 11, further comprising:
acquiring new second 2D B-frame images until a convergence to a target tolerance is achieved.

14. A computer-readable storage device storing non-volatile computer-executable instructions, which, when executed, cause an ultrasound system to:
(i) transmit an imaging ultrasound toward a target tissue;
(ii) receive ultrasound echoes of the imaging ultrasound using a first set of receiving beamformer parameters;
(iii) compose a first two-dimensional (2D) B-mode image of the target tissue based on received ultrasound echoes of the imaging ultrasound;
(iv) transmit a therapy ultrasound toward a target tissue using a second set of transmitting beamformer parameters;
(v) receive ultrasound echoes of the therapy ultrasound using the first set of receiving beamformer parameters;
(vi) compose a second 2D B-mode image of the target tissue based on received ultrasound echoes of the therapy ultrasound; and
(vii) compare the first 2D B-mode image of the target tissue with the second 2D B-mode image of the target tissue.

15. The computer-readable storage device of claim 14, wherein the instructions further cause the ultrasound system to:
determine whether a match between the first 2D B-mode image and the second 2D B-mode image is within a predetermined threshold match;
if the match is within the predetermined threshold match, transmit additional therapy ultrasound toward the target tissue using the second set of transmitting beamformer parameters; and
compare the first 2D B-mode image of the target tissue with additional second 2D B-mode image of the target tissue.

16. The computer-readable storage device of claim 14, wherein the instructions further cause the ultrasound system to:
determine whether a match between the first 2D B-mode image and the second 2D B-mode image is within a predetermined threshold match;
if the match is outside of the predetermined threshold match, adjust the second set of transmitting beamformer parameters;
transmit the therapy ultrasound toward the target tissue using the adjusted second set of transmitting beamformer parameters; and
compare the first 2D B-mode image of the target tissue with additional second 2D B-mode image of the target tissue.

17. The computer-readable storage device of claim 16, wherein adjusting the second set of transmitting beamformer parameters is performed by an artificial intelligence (AI) engine.

18. The computer-readable storage device of claim 17, wherein the AI engine utilizes transducer template beam patterns as training sets to produce new element-by-element time delays that minimize differences between the second 2D B-frame and the first 2D B-frame.

19. The computer-readable storage device of claim 14, wherein:
the imaging ultrasound is transmitted by an imaging transducer;
the ultrasound echoes of the imaging ultrasound are received by the imaging transducer;
the therapy ultrasound is transmitted by a therapy transducer; and
the ultrasound echoes of the therapy transducer are received by the imaging transducer.

20. The computer-readable storage device of claim 14, wherein:
the imaging ultrasound and the therapy ultrasound are transmitted and received by a same transducer which alternately executes roles of the imaging transducer and the therapy transducer.
